(19) United States
(12) Patent Application Publication
(10) Pub. No.: US 2026/0232226 A1
(43) Pub. Date: Aug. 13, 2026

(54) Multimodal Characterisation of Tinnitus

(71) Applicant: THE BIONICS INSTITUTE OF AUSTRALIA, Victoria (AU)

(72) Inventors: Mehrnaz Shoushtarian, East Melbourne (AU); James Fallon, East Melbourne (AU); Michelle Monica Gandol Bravo, East Melbourne (AU); Shreyasi Datta, East Melbourne (AU)

(21) Appl. No.: 19/154,259

(22) PCT Filed: Feb. 6, 2024
(86) PCT No.: PCT/AU2024/050070

§ 371 Date: Aug. 6, 2025

(30) Foreign Application Priority Data
Feb. 6, 2023 (AU) 2023900282

(51) Int. Cl. A61B 5/12; A61B 5/00; A61B 5/024; A61B 5/0533

(52) U.S. Cl. CPC A61B 5/128; A61B 5/0042; A61B 5/0075; A61B 5/024; A61B 5/0533; A61B 5/7264

(57) ABSTRACT

Disclosed is a method for characterising tinnitus in a subject. The method comprises receiving data comprising cortical activity data indicative of cortical activity in one or more regions of the subject's brain, and cardiac activity data indicative of cardiac activity in the subject, at a processor device. The cortical activity data is derived from one or more functional near-infrared spectroscopy (fNIRS) signals. The received data is processed using the processor device by inputting feature values into a model, where the feature values include one or more features of the cortical activity data and one or more features of the cardiac activity data. The model is configured to provide one or more classification results based on the feature values, the classification results being indicative of at least one characteristic of tinnitus in the subject. Also disclosed is a system for applying the disclosed method.

### CROSS REFERENCE TO RELATED APPLICATIONS

This application claims priority to Australian provisional patent application No. 2023900282, filed 6 Feb. 2023, which is hereby incorporated by reference in its entirety.

### TECHNICAL FIELD

The present disclosure relates to methods and systems for characterisation of tinnitus.

### BACKGROUND

Tinnitus is a medical condition characterised by perception of sounds that are not present externally. Chronic tinnitus is a debilitating condition which affects 6-20% of adults and can severely impact their quality of life. Approximately 20% of adults with tinnitus experience it in a severe form, along with associated symptoms such as depression, cognitive dysfunction and stress.

Despite its wide prevalence, there is currently no clinically used objective test for assessment of tinnitus. In general, clinical assessment of tinnitus relies on subjective feedback from individuals, which may be inaccurate.

Functional near infrared spectroscopy (fNIRS, also known as Optical Tomography) is a non-invasive brain imaging technique which uses near-infrared light to monitor changes in oxygenated haemoglobin (HbO) and de-oxygenated haemoglobin (HbR) concentrations using a montage of light sources and detectors. Cortical brain activity may be inferred from fNIRS signals indicative of changes in HbO concentration and/or signals indicative of changes in HbR concentration in the subject's brain.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

According to one aspect of the present disclosure, there is provided a method for characterising tinnitus in a subject, the method comprising:
receiving data at a processor device, the received data comprising:
   cortical activity data, indicative of cortical activity in one or more regions of the subject's brain, the cortical activity data derived from one or more functional near-infrared spectroscopy (fNIRS) signals; and
   cardiac activity data, indicative of cardiac activity in the subject;
processing the received data using the processor device, the processing comprising:
   inputting, into a model, feature values including one or more features of the cortical activity data and one or more features of the cardiac activity data,
   wherein the model is configured to provide one or more classification results based on the feature values, the one or more classification results being indicative of at least one characteristic of tinnitus in the subject.

The cardiac activity data may be derived from one or more signals provided by one or more electrical sensors and/or one or more optical sensors. The one or more electrical sensors and/or one or more optical sensors may be separate to a source of the fNIRS signals. For example, the cardiac activity data may be derived from signals from a wearable sensor device. Alternatively, or additionally, the cardiac activity data may be derived from the one or more fNIRS signals. For example, the cardiac activity data may comprise a cardiac signal component of one or more of the fNIRS signals, which cardiac signal component may be different from a cortical activity component of the one or more of the fNIRS signals.

The feature values may include one or more features of heart rate data. Heart rate (also known as pulse rate) may be understood as the frequency of contractions of the subject' heart as measured by the number of contractions over a predetermined time interval. Heart rate may be measured in beats per minute (bpm), for example. The cardiac activity data may comprise the heart rate data or processing the received data may comprise determining heart rate data based on the cardiac activity data.

The feature values may include one or more features of heart rate variability (HRV) data. Heart rate variability may be understood as a measure of variations in the inter-beat interval (i.e. the time between consecutive heartbeats). The cardiac activity data may comprise the heart rate variability data or processing the received data may comprise determining heart rate variability data based on the cardiac activity data.

In some embodiments, the fNIRS signals may be indicative of activity in regions of the subject's brain including one or more of the frontal, left temporal, right temporal and occipital cortical regions. In some embodiments, the regions of the subject's brain include each of the frontal, left temporal, right temporal and occipital cortical regions of the subject's brain. In some embodiments, the fNIRS signals may be indicative of cardiac activity in the subject.

In some embodiments, the classification results may include a presence or absence of tinnitus in the subject. Additionally or alternatively, the classification results may include a severity rating of tinnitus in the subject. In some embodiments, the severity rating may categorise the tinnitus as either slight to mild tinnitus or moderate to severe tinnitus. In other embodiments, the severity rating may be selected from a greater number of categories. For example, the possible ratings may include slight tinnitus, mild tinnitus, moderate tinnitus and severe tinnitus, or other categories. In some embodiments, the severity rating may be selected from a range of severities. For example, the severity rating may be expressed on a numerical scale.

In some embodiments, the model may provide a quantification (e.g., as perceived by the subject) of loudness of the tinnitus and/or of annoyance produced by the tinnitus. Providing a quantification of loudness and/or of annoyance may be useful, for example, in assessing the impact of the tinnitus on the quality of life of the subject. Providing a quantification of loudness and/or annoyance may also be useful in developing treatments for tinnitus and in defining parameters for assessing the relative success of such treatments.

Feature values may be extracted from the received data using one or more methods. In some embodiments, feature values are extracted from the received data using Information Gain. Information Gain is a measure of entropy in the data, enabling identification of channels and HbO/HbR features with the most relevant information for classification. Information Gain may be used, for example, to select the most relevant features by ranking them based on their weight or importance in classification. In other embodiments, alternative features selection methods may be used. For example, the features values may be extracted using one or more of Gini index, SVM (Support Vector Machine) weight, wrapper method, or other suitable methods (for example, different entropy methods).

In some embodiments, the model may comprise an algorithm. In some embodiments, the model may comprise a trained model. The model may have been trained with an artificial intelligence (AI) algorithm based on a previous one or more feature values, for example. The previous one or more feature values may have been mapped to subjective measures of characteristics of tinnitus.

The model may provide classification results using a classification algorithm. The classification algorithm may include one or more of the following: Nave Bayes; K-nearest neighbour (KNN); Rule Induction; and Artificial Neural Networks (ANN), support vector machine (SVM with, for example, e.g. linear and Gaussian kernels) and decision trees. In other embodiments, alternative classification algorithms, or customised algorithms, may be used. In some embodiments, the classification algorithm may be multi-level hierarchical classification with binary classifiers at each level. This method of classification may provide advantages compared to a single-level classifier, due to increased flexibility in selecting the most relevant features for each component binary classification module. This method may provide improved classification performance compared to single level multiclass classification.

In some embodiments, the method may comprise applying a therapy for treating tinnitus and, through the processing of the received data, detecting a change in the one or more characteristics of the tinnitus as a result of applying the therapy. Changes detected as a result of applying therapy may be used to make an assessment of the effectiveness, or otherwise, of the applied therapy. In some embodiments, the model may be configured to provide a prognostic measure indicative of whether a proposed therapy for treating tinnitus is likely to be effective for treating tinnitus in the subject.

In some embodiments, the method may comprise determining a quality of each fNIRS signal and removing signals of inadequate quality prior to the processing of the received data. The quality of each fNIRS signal may be determined based on a level of signal gain, for example. Channels with high gains may be indicative of inadequate detected light intensity. Alternatively, or additionally, the quality of each fNIRS signal may be determined based on a level of cardiac signal content (as indicative of a level of contact between recording optodes of an fNIRS device and the scalp of the subject). In some embodiments, the fNIRS signals may be subject to pre-processing steps to improve signal quality prior to the processing of the received data. In some embodiments, an algorithm (e.g. a movement reduction algorithm) may be used to detect and remove motion artefacts from the fNIRS signals prior to the processing of the received data. Additionally or alternatively, signals containing movement artefacts in excess of a maximum threshold may be determined to be of inadequate quality and removed prior to processing the received data.

In any aspect herein, the fNIRS signals may comprise a plurality of signals obtained using an fNIRS system that measures at least a level of cortical activity in the one or more regions of the subject's brain. The fNIRS system may additionally measure one or more characteristics of cardiac activity in the subject. The fNIRS system may comprise a multi-channel fNIRS system. Each channel may be defined by a source-detector pair. The fNIRS system may comprise a continuous-wave fNIRS system.

In some embodiments, the fNIRS signals may comprise signals from a plurality of channels positioned over each of the respective regions of the subject's brain. In some embodiments, at least one fNIRS signal may be indicative of systemic signals from at least one superficial layer of the subject's head, including the scalp and/or the skull. Such systemic signals may be used in pre-processing of the received data, to remove unwanted artefacts or noise, for example.

In some embodiments, the received data may comprise resting-state data obtained while the subject is at rest. The resting-state data may comprise resting-state cortical activity data and/or resting-state cardiac activity data. The resting-state data may comprise fNIRS signals obtained during a period in which no stimulus (such as an auditory or visual stimulus) is delivered to the subject, and/or prior to delivery of any stimulus (such as an auditory or visual stimulus) to the subject. In one example, the resting-state data comprises data, including fNIRS signals, obtained over a period of about 6 minutes. In other embodiments, the resting-state data may be obtained over a period having a shorter or a longer duration.

The resting-state data may comprise fNIRS signals indicative of cortical activity in two or more regions of the subject's brain while the subject is at rest. In such embodiments, processing the received data may comprise determining at least one resting-state functional connectivity measure between the at least two regions of the subject's brain based on the resting-state data. Resting-state functional connectivity is a measure of (or is indicative of) a level of coordination between different neural populations. The one or more feature values may include one or more features of the at least one resting-state functional connectivity measure.

In some embodiments, the resting-state functional connectivity measure may be determined from the resting-state data using a Seed Analysis method. The Seed Analysis method may comprise selecting at least one region of the subject's brain as a seed region, and correlating a seed fNIRS signal from the seed region with at least one fNIRS signal from at least one other region of the subject's brain. For example, in some embodiments, a temporal cortex of the subject's brain may be selected as the seed region. In other embodiments, other regions of the subject's brain may be selected as the seed region. The seed fNIRS signal from the seed region may be correlated with at least one fNIRS signal from another region of the subject's brain. For example, the seed fNIRS signal may be correlated with a frontal cortical region, occipital cortical region and/or contralateral temporal region of the subject's brain, or other region of the subject's brain.

In some embodiments, more than one seed region may be selected. For example, seed regions on left and right sides of the subject's brain may be selected in order to determine respective left and right resting-state functional connectivity measures. In some such embodiments, both a left and a right temporal cortex of the subject's brain are selected as respective left and right seed regions.

In some embodiments, the seed fNIRS signal may comprise the average of a plurality of fNIRS signals from the seed region. The seed fNIRS signal may be correlated with values obtained for channels in another region of the subject's brain. The correlations with each channel may be assessed individually or averaged (for example, across a region). The correlation may be performed on fNIRS signals comprising HbO concentration measurements and/or fNIRS signals comprising HbR concentration measurements.

In other embodiments, other connectivity analysis methods may be used, additionally or alternatively, to determine the at least one resting-state functional connectivity measure. For example, other time-domain and/or frequency-domain methods of analysing resting-state connectivity may be used. For example, the resting-state functional connectivity measure may be determined using independent component analysis (ICA) and/or graph connectivity analysis.

In some embodiments, processing the received data may comprise determining an average resting heart rate, based on the resting-state cardiac activity data. Processing the received data may comprise determining heart-rate variability based on the resting-state cardiac activity data. In such embodiments, the feature values may include the average resting heart rate and/or the heart rate variability. Heart-rate variability may be determined using one or more entropy methods. In some embodiments, heart-rate variability may be determined using entropy profiling. In other embodiments, heart-rate variability may be determined using sample entropy or fuzzy entropy. Alternatively or additionally, heart rate variability may be determined by one or more other suitable time-domain methods and/or frequency-domain methods. For example, heart rate variability may be determined using methods such as Poincaré plots where inter-beat intervals are translated to a geometric shape, from which heart rate variability information is extracted.

In some embodiments, the received data may comprise evoked response data. The evoked response data may include evoked cortical activity data and/or evoked cardiac activity data. The evoked response data may be obtained during and/or following delivery of at least one stimulus to the subject. For example, the evoked response data may comprise fNIRS signals indicative of cortical activity in at least one region of the subject's brain during and/or after delivery of at least one stimulus to the subject and cardiac data indicative of cardiac activity during and/or following delivery of the stimulus to the subject. The fNIRS signals may be indicative of cortical activity and/or cardiac activity resulting from delivery of the at least one stimulus. In such embodiments, the feature values may include one or more features of the evoked response data.

In such embodiments, the one or more feature values may include one or more features of the evoked cortical activity data. In some embodiments, the feature values may include one or more amplitudes of the evoked cortical activity data. For example, the feature values may include a peak amplitude, an absolute peak amplitude, or a mean amplitude of the evoked cortical activity data.

Alternatively or additionally, the feature values may include alternative features of the evoked cortical activity data. For example, the feature values may include one or more of: a variance, an area under the curve, an absolute area under the curve, a peak power amplitude, an entropy of the waveform; a temporal content of the waveform; and a spectral content of the waveform of the evoked cortical activity data. As another example, the feature values may include ‘principal components’ of the response waveforms of the evoked cortical activity data, calculated using Principal Component Analysis. In Principal Component Analysis, all the evoked responses to one or more stimuli are considered and the principal components of the responses are calculated. A number of these components (for example, the first ten components, or those covering the majority of the variability in the data) may then be used as feature values. In some cases, the feature value may be determined across a predefined time period. The time period may be, for example, across the duration of the stimulus, across 0-5 seconds after the start of an auditory stimulus, across 10-15 seconds after the start of a visual stimulus, or other time periods as appropriate.

In some embodiments, a general linear model (GLM) may be used to compare the evoked cortical activity data to a model of typical hemodynamic response function. From this comparison, a coefficient may be generated which my indicate whether a response is detected. The coefficient may function as a feature value of the evoked cortical activity data. Additionally or alternatively, feature values of evoked cortical activity data may be compared to corresponding features of the model hemodynamic response to determine a correlation between the evoked cortical activity data and a typical hemodynamic response.

Processing the evoked cardiac activity data may comprise determining a percentage change in heart rate during and/or following delivery of the stimulus to the subject, based on the evoked cardiac activity data. The feature values may include the percentage change in heart rate. Processing the evoked cardiac activity data may comprise determining an evoked heart rate variability during and/or following delivery of the stimulus to the subject, based on the evoked cardiac activity data. The feature values may include the evoked heart rate variability.

The at least one stimulus may comprise an auditory stimulus. In some embodiments, the auditory stimulus may comprise pink noise. In some embodiments, the auditory stimulus may be provided at a Sound Pressure Level of about 65 dB. The auditory stimulus may be provided at a predetermined level above hearing threshold. Additionally or alternatively, other forms of auditory stimulus may be used. The auditory stimulus may be configured to evoke a strong cortical response in the subject.

The at least one stimulus may comprise a visual stimulus. The visual stimulus may be configured to evoke a strong cortical response in the subject. In some embodiments, the visual stimulus may comprise a display of a pattern, e.g., a black and white pattern, such as a checkerboard pattern. The checkerboard pattern may comprise a radial configuration including concentric rings divided into sectors, wherein neighbouring sectors are of opposite colour. The visual stimulus may comprise repeated reversal (or flickering) of the pattern. The reversal of the pattern may be applied at a temporal frequency of about 7.5 Hz (that is, about 15 reversals per second). Additionally or alternatively, other forms of visual stimulus may be used.

In some embodiments, the at least one stimulus may comprise a plurality of discrete stimuli delivered to the subject in a sequence. The at least one stimulus may comprise a plurality of auditory stimuli, a plurality of visual stimuli or a combination of at least one auditory stimulus and at least one visual stimulus. In some embodiments, the method may comprise providing a plurality of auditory stimuli and a plurality of visual stimuli in a sequence. For example, the plurality of auditory stimuli and the plurality of visual stimuli may be provided in an alternating sequence (e.g. alternating one auditory stimuli with one visual stimuli, or alternating one or more auditory stimuli with one or more visual stimuli). Alternatively, the plurality of auditory stimuli and the plurality of visual stimuli may be applied in a different predetermined sequence, or in a substantially randomised order. In some embodiments, the plurality of auditory stimuli and the plurality of visual stimuli may be applied (e.g. pseudo-randomly) so that there is no more than two applications of the same stimulus type (e.g. visual or auditory) in a row.

Each stimulus may have a set duration. For example, in some embodiments, each stimulus may have a duration of about 15 seconds. However, stimuli of other durations may be used. In some embodiments, each stimulus may have a substantially identical duration. In other embodiments, the duration of the stimuli may vary.

Each stimulus may be followed by a rest period in which no stimulus is provided. Each rest period may have a predetermined duration. For example, each rest period may have a duration of between about 20 seconds to about 30 seconds, or more. For example, each rest period may have a duration of about 20 seconds, about 25 seconds, about 30 seconds, or more. In some embodiments, the rest periods may each have a substantially identical duration. In other embodiments, the duration of the rest periods may vary. In some embodiments, the duration of the rest periods may be randomly selected. According to one aspect of the present disclosure, there is provided a non-transitory machine readable storage medium comprising instructions configured to cause a processor device to execute methods according to the present disclosure.

According to another aspect of the present disclosure, there is provided a system for characterising tinnitus in a subject, the system comprising:
a processor device, configured to:
   receive data, the received data comprising:
      cortical activity data, indicative of cortical activity in one or more regions of the subject's brain, the cortical activity data derived from one or more functional near-infrared spectroscopy (fNIRS) signals; and
      cardiac activity data, indicative of cardiac activity in the subject; and
   process the received data, wherein the processing comprises:
      inputting, into a model, feature values including one or more features of the cortical activity data and one or more features of the cardiac activity data,
      wherein the model is configured to provide one or more classification results based on the feature values, the one or more classification results being indicative of at least one characteristic of tinnitus in the subject.

In general, the system may be configured to carry out any one or more of the methods steps described in embodiments above, including in relation to pre-processing of the received data, processing of the received data, or otherwise. The model may be a trained model, trained with an artificial intelligence (AI) algorithm, and may include one or more of the features as described in relation to the model above.

The system may comprise a fNIRS system configured to provide the fNIRS signals. The fNIRS system may be configured to measure at least a level of cortical activity in at least two regions of the subject's brain. The fNIRS system may comprise a multi-channel system. Each channel may be defined by a source-detector pair. For example, the fNIRS system may comprise a plurality of channels configured to be positioned over each of the at least two regions of the subject's brain. In one example, the fNIRS system comprises a plurality of channels configured to be positioned over each of the frontal, left and right temporal and occipital regions of the subject's brain.

In some embodiments, the cardiac activity data may be derived from the one or more fNIRS signals. Additionally or alternatively, the cardiac activity data may be derived from one or more signals provided by one or more electrical sensors and/or one or more optical sensors. The one or more electrical sensors and/or one or more optical sensors may be part of the fNIRS system. Alternatively, the one or more electrical sensors and/or one or more optical sensors may be separate from the fNIRS system. The cardiac data may be derived from signals obtained from a wearable sensor device, for example. In some embodiments, the one or more sensors may comprise a photoplethysmography sensor. The cardiac data may be derived from signals provided by the photoplethysmography sensor.

The system may comprise an auditory stimulator configured to deliver an auditory stimulus to the subject. The system may comprise a visual stimulator configured to deliver a visual stimulus to the subject.

In some embodiments, the system may comprise a display configured to display the one or more classification results. In some embodiments, the system may comprise a user input module.

Generally, it will be recognised that the processor device according to embodiments of the present disclosure may comprise one or more processing components for carrying out processing steps according to the present disclosure and may also include one or more storage devices, for storing data such as the resting-state data and/or evoked response data. The processing components and/or storage devices may be at one location or distributed across multiple locations and interconnected by one or more communication links.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

By way of example only, embodiments of the present disclosure are now described with reference to the accompanying Figures in which:

FIG. 1 shows a flowchart of steps in a method of characterising tinnitus in a subject, according to one embodiment of the present disclosure;

FIG. 2 shows a flowchart of steps in a method of characterising tinnitus in a subject, according to another embodiment of the present disclosure;

FIG. 3 shows a timeline of an example test session used to obtain data according to the method of FIG. 2;

FIG. 4 shows a schematic diagram of an example system for characterising tinnitus according to embodiments of the present disclosure;

FIG. 5 shows a detail of a source-detector channel layout in a multi-channel fNIRS system in the system of FIG. 4;

FIGS. 6A and 6B illustrate a process of data collection and analysis in one example of the present disclosure;

FIG. 7 illustrates oxygenated haemoglobin (HbO) evoked cortical activity data in response to auditory stimulation in tinnitus and controls;

FIG. 8 illustrates deoxygenated haemoglobin (HbR) evoked cortical activity data in response to auditory stimulation in tinnitus and controls;

FIG. 9 illustrates HbO evoked cortical activity data in response to visual stimulation in tinnitus and controls;

FIG. 10 illustrates HbR evoked cortical activity data in response to visual stimulation in tinnitus and controls;

FIG. 11A shows left seed HbO-derived whitened correlations for subjects with tinnitus and controls;

FIG. 11B shows right seed HbO-derived whitened correlations for subjects with tinnitus and controls;

FIG. 12A shows an example of feature selection using group comparisons and feed forward selection for subjects with tinnitus and controls;

FIG. 12B shows selected features on a source-detector channel layout for comparing subjects with tinnitus and controls;

FIG. 12C shows selected features on a source-detector channel layout for comparing subjects grouped by low Tinnitus Handicap Inventory scores and high Tinnitus Handicap Inventory scores;

FIG. 12D shows selected features on a source-detector channel layout for comparing subjects grouped by low loudness scores and high loudness scores;

FIG. 12E shows selected features on a source-detector channel layout for comparing subjects grouped by low annoyance scores and high annoyance scores;

FIG. 13 shows electro-dermal activity (Panel A) and percentage change in heart-rate (Panel B) in response to stimuli delivered to the subject, with dashed lines indicating stimulus onset and offset times;

FIG. 14 shows scores from a perceived stress questionnaire (PSQ) index in subjects with tinnitus and controls;

FIG. 15A compares a heart rate variability measure, SD1, for tinnitus subjects grouped by low annoyance and high annoyance scores;

FIG. 15B compares heart rate (HR) (left-hand panel) and percentage change in heart rate (right-hand panel) in response to visual stimulus presented to the subject, for subjects grouped by low and high perceived stress questionnaire (PSQ) index scores;

FIG. 16 compares three entropy methods for characterising heart rate variability (HRV), correlated against perceived stress questionnaire (PSQ) index scores;

FIG. 17 compares average sample entropy to Tinnitus Handicap Inventory (THI) scores;

FIG. 18 illustrates HbO evoked cortical activity data in response to auditory stimulation in tinnitus and controls;

FIG. 19 illustrates HbO evoked cortical activity data in response to visual stimulation in tinnitus and controls; and

FIG. 20 compares three entropy methods for characterising heart rate variability (HRV), correlated against perceived stress questionnaire (PSQ) index scores.

### DETAILED DESCRIPTION

Methods for characterising tinnitus in a subject according to embodiments of the present disclosure are now described.

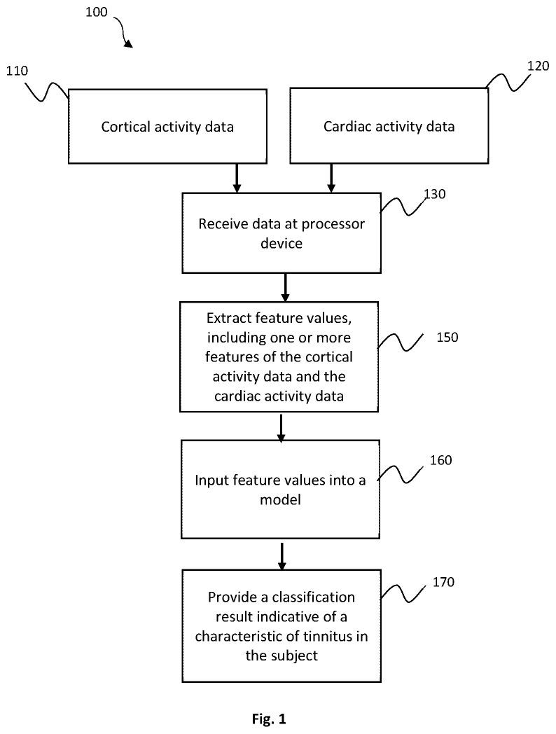

Referring to flowchart 100 of FIG. 1, a method of characterising tinnitus in a subject according to an embodiment of the present disclosure is shown. The method comprises receiving data 110, 120 at a processor device 130. The received data comprises cortical activity data 110 and cardiac data 120. The cortical activity data 110 comprises fNIRS signals indicative of cortical activity in one or more regions of the subject's brain.

The fNIRS signals indicative of cortical activity in one or more regions of the subject's brain may include fNIRS signals indicative of changes in deoxyhaemoglobin (HbR) concentration and/or oxyhaemoglobin (HbO) concentration in cortical regions of the subject's brain. Cortical brain activity in the measured regions may be inferred from these measurements. The fNIRS signals may also be indicative of other physiological parameters. For example, the fNIRS signals may contain a cardiac signal component indicative of cardiac activity in the subject. In such cases, the cardiac data 120 may be derived from the fNIRS signals. Alternatively or additionally, the cardiac data 120 may be derived from signals from one or more sensors, separate to the fNIRS signals.

The fNIRS signals (and/or cardiac activity signal, if derived separately from the fNIRS signals) may be filtered, down-sampled, or otherwise pre-processed. In some embodiments, the fNIRS signals may be pre-processed to remove signals of inadequate quality. For example, the quality of each signal may be determined and signals of inadequate quality removed from the data prior to further processing of the received data. Alternatively, or additionally, undesirable artefacts in the fNIRS signals (due to motion or other interference, for example) may be filtered from the signals prior to further processing of the received data. In other embodiments, for example, embodiments employing AI algorithms, signals of inadequate quality and/or undesirable artefacts may be retained in the signals and the algorithm trained to disregard data from these signals/artefacts.

In some cases, quality of the fNIRS signals may be determined by checking for cardiac signal content of the fNIRS signals, using a scalp coupling index (SCI) for example. This provides an indication of the degree of contact between optodes (detectors) and the scalp. Signals from optodes with good skin contact will have a prominent cardiac waveform and may be of higher quality. Where cardiac activity data is obtained from signals separate to the fNIRS signals, the cardiac signal content of the fNIRS signals may be disregarded. However, in other embodiments the cardiac activity data 120 may be derived from the cardiac signal content of the fNIRS signals.

One or more feature values 150, including one or more features extracted from the cortical activity data 110 and the cardiac activity data 120, are then input into a model 160.

The model 160 may comprise a trained model. For example, the model 160 may have been trained with an artificial intelligence (AI) algorithm based on a previous one or more feature values mapped to subjective measures of characteristics of tinnitus. For example, the model 160 may have been trained using feature values derived from previously obtained cortical activity data and/or cardiac activity data. The model 160 may be configured to provide one or more classification results 170 based on the one or more feature values 160, the classification results 170 being indicative of at least one characteristic of tinnitus in the subject. The classification results 170 may include, for example, a presence or absence of tinnitus in the subject, a severity of tinnitus in the subject, a quantification of loudness of the tinnitus and/or a quantification of annoyance produced by the tinnitus. The classification results may be determined using one or more suitable classification algorithms, for example, Naïve Bayes, K-nearest neighbour (KNN), Rule Induction, Artificial Neural Networks (ANN), multi-level hierarchical classification, support vector machine (SVM, with e.g. linear and gaussian kernels) and/or decision trees.

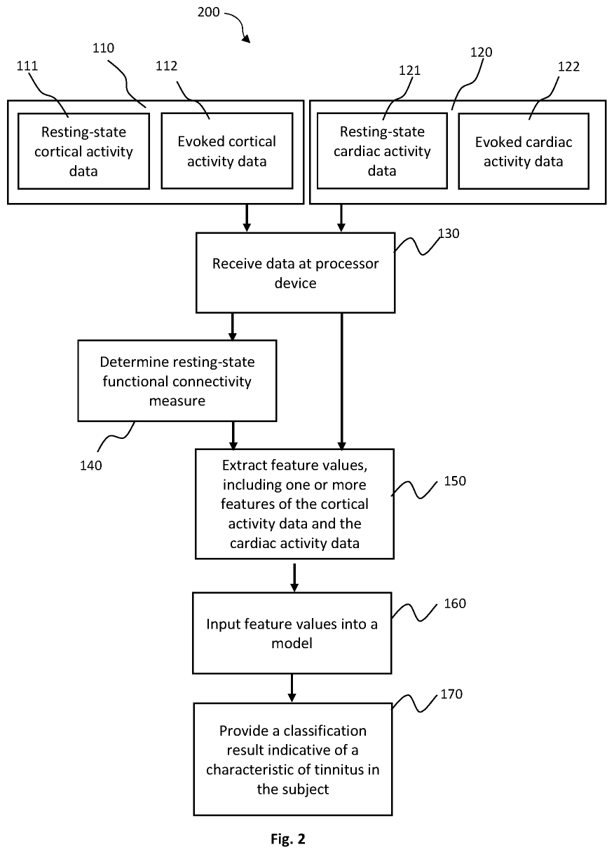

As shown in FIG. 2, the cortical activity data 110 and the cardiac activity data 120 may each comprise resting-state data and/or evoked response data, received at the processor device 130. For example, the cortical activity data 110 may comprise resting-state cortical activity data 111 and/or evoked cortical activity data 112. The cardiac activity data 120 may comprise resting-state cardiac activity data 121 and/or evoked cardiac activity data 122.

The resting-state data 111, 121 may be obtained over a period while the subject is at rest, and/or prior to delivery of any stimulus to the subject.

The evoked response data 112, 122 may comprise signals indicative of a response to at least one stimulus. The method may comprise steps of delivering at least one stimulus to the subject and recording the evoked response data 112, 122. The evoked response data 112, 122 may correspond to fNIRS signals (and optionally additional signals indicative of cardiac activity) recorded during and/or after delivery of the stimulus. For example, the evoked cortical activity data 112 may comprise fNIRS signals indicative at least of cortical activity in at least one region of the subject's brain resulting from at least one stimulus delivered to the subject.

The one or more feature values 150 may include one or more of: the resting-state cortical activity data 111, the resting-state cardiac activity data 121, the evoked cortical activity data 112 and/or the evoked cardiac activity data 122.

The one or more feature values 150 of the resting-state cortical activity data 111 may include at least one resting-state functional connectivity measure 140. Based on the resting-state cortical activity data 111 (after any pre-processing steps have been applied), at least one resting-state functional connectivity measure 140 is determined between at least two regions of the subject's brain. The measure of resting-state functional connectivity 140 may be determined using a Seed Analysis method, for example, although other methods of determining connectivity may be used as appropriate.

The one or more feature values 150 of the evoked cortical activity data 112 may include a peak amplitude, an absolute peak amplitude, or a mean amplitude across a predefined time period. Alternatively or additionally, the feature values 150 may include one or more of: a variance, an area under the curve, an absolute area under the curve, a peak power amplitude, an entropy of the waveform; a temporal content of the waveform; a spectral content of the waveform; ‘principal components’ of the response waveforms (for example, calculated using Principal Component Analysis), or other features of the evoked cortical activity data 112. The feature values may be different for auditory and visual evoked cortical activity data 112, depending on the model 160 and the classification algorithm used.

The one or more feature values 150 of the resting-state cardiac activity data 121 may include a heart rate, for example an average heart rate from at least a portion of the resting-state cardiac activity data 121.

The one or more feature values 150 of the evoked cardiac activity data 122 may include a change in heart rate over a duration of applying the stimulus, and/or over a predetermined period following cessation of applying the stimulus. The one or more feature values 150 of the evoked cardiac activity data 122 may include a heart rate variability over a duration of applying the stimulus, and/or over a predetermined period following cessation of applying the stimulus. The model 160 may determine an optimal combination of channels, feature values and classification algorithms to provide the classification results.

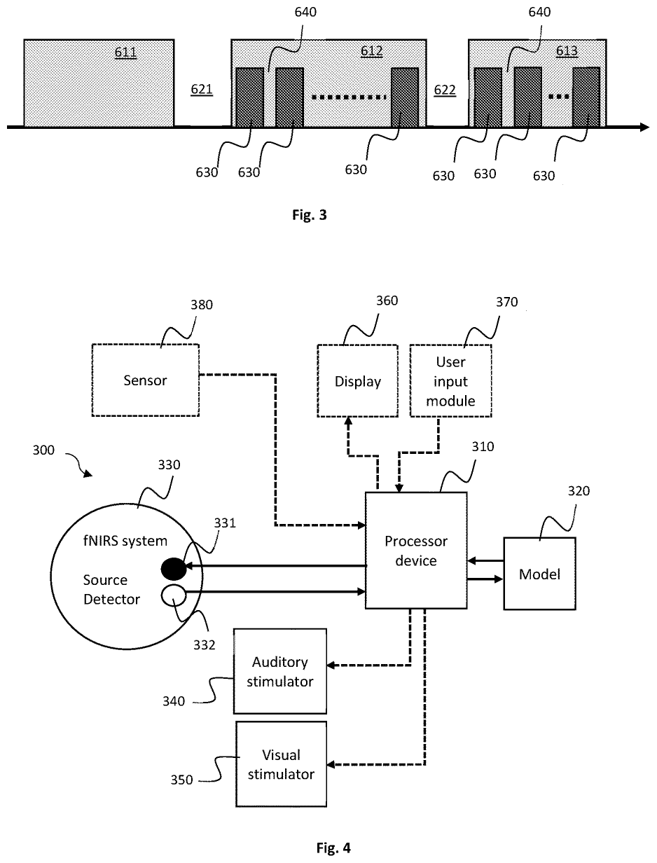

FIG. 3 shows an illustrative timeline of a test session. The illustrated test session consisted of three recording sessions 611, 612, 613 with short rest breaks 621, 622 given in between. In some embodiments, the rest breaks may be of about 3-5 minutes duration, although other durations may be used. The first recording session 611 included resting-state recording. The resting-state recording may have a predetermined duration. In one example, a six-minute resting-state recording is used, although recordings of other durations may also be suitable. During this recording, the subject was instructed to sit still with their eyes closed but not fall asleep. No auditory or visual stimulus was provided to the subject during the session 611.

The second and third recording sessions 612, 613 each included a series of evoked response recordings. In these recording sessions 612, 613, a plurality of 15-second stimuli 630 were delivered to the subject in sequence. In this example, discrete auditory and visual stimuli 630 were delivered in randomised order, with no more than two of the same stimulus type in a row. However, other arrangements and durations of stimuli 630 may be used.

Each stimulus 630 was followed by a non-stimulus interval period 640. In one example, the non-stimulus interval was 20 or 25 seconds, chosen at random. In other embodiments, alternate durations of non-stimulus intervals may be used. In general, the duration of each non-stimulus time period 640 may be selected to allow enough time for any evoked response to subside and the cortical activity to revert to baseline. The dotted lines in FIG. 3 indicate repetition of the pattern of stimulus delivery. Each stimulus type (i.e. auditory or visual) may be repeated a predetermined number of times. In one example, each stimulus type was repeated 10 times in total (six in the second recording and four in the third recording with a break in between the two recordings), although in other examples an alternative number of stimuli may be used. The total recording time (excluding breaks) was approximately 20 minutes.

Data (e.g., from fNIRS signals and, optionally, a separate sensor for detecting cardiac activity) may be recorded continuously during each recording session. For the evoked response recording sessions 612, 613, the continuous data recording was later correlated with the time of delivery of each of the stimuli 630 to extract portions of the data corresponding to each evoked response.

FIG. 4 shows a schematic diagram of an example system for characterising tinnitus in a subject according to embodiments of the present disclosure. The system comprises a processor device 310 which is configured to receive data, including resting-state cardiac activity data, resting-state cortical activity data, evoked cardiac activity data and/or evoked cortical activity data. The received data may comprise signals, such as fNIRS signals indicative of cortical brain activity in the subject and/or signals indicative of cardiac activity in the subject.

The processor device 310 is configured to process the resting-state data to determine at least one resting-state functional connectivity measure between the at least two regions of the subject's brain based on the resting-state data.

The processor device 310 is further configured to input one or more feature values of at least one resting-state functional connectivity measure, and/or one or more feature values of the resting-state cardiac activity data, resting-state cortical activity data, evoked cardiac activity data and/or evoked cortical activity data, into a model 320.

The model 320 is configured to provide one or more classification results based on the one or more feature values, the one or more classification results being indicative of at least one characteristic of tinnitus in the subject. The model 320 may be a trained model or otherwise. For example, the model 320 may have been trained with an artificial intelligence (AI) algorithm.

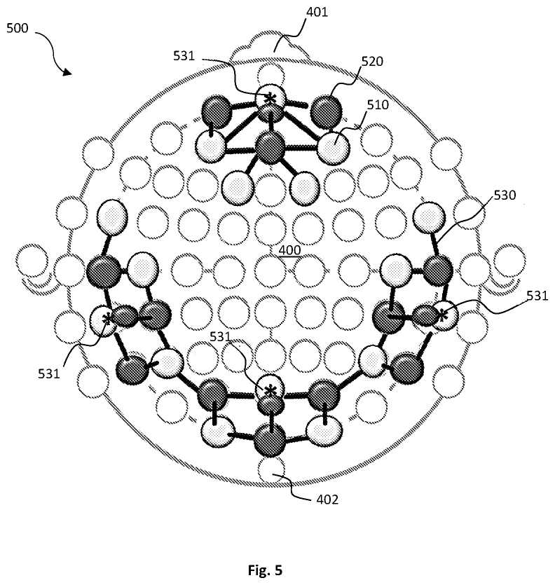

Referring again to FIG. 4, the fNIRS signals may be obtained using an fNIRS system 330, where the fNIRS system 330 is configured to measure a level of cortical activity in at least two regions of the subject's brain. The processor device 310 may receive the data comprising fNIRS signals directly from the fNIRS system 330, as shown in FIG. 3. Alternatively, or additionally, fNIRS signal data may be initially received and stored at an intermediate data collection device and later received by the processor device 310. The fNIRS system 330 may comprise a multi-channel fNIRS system, wherein each fNIRS channel is defined by a source-detector pair. A single, representative fNIRS channel is illustrated in FIG. 3 by source 331 and detector 332. In some embodiments, the fNIRS system 330 may comprise a plurality of channels configured to be positioned over each respective region of the subject's brain. In one such embodiment, as illustrated in FIG. 5 and discussed in more detail in Example 1 below, the fNIRS system 330 comprises a plurality of channels configured to be positioned over each of the frontal, left and right temporal and occipital regions of the subject's brain.

In some embodiments, the cardiac activity data may be derived from the fNIRS signals obtained from the fNIRS system 330. Additionally or alternatively, the system 300 may comprise a sensor 380 for providing a signal indicative of cardiac activity in the subject. The sensor may comprise a photoplethysmography sensor, for example. In other embodiments, the sensor 380 may comprise an alternative sensor suitable for detecting cardiac activity of the subject.

The processor device 310 may directly or indirectly control the operation of the fNIRS system 330. For example, the processor device 310 may include a light output module for controlling each source 331 of the fNIRS system and a data input module for receiving fNIRS signals from each detector 332 of the fNIRS system 330. Alternatively, the fNIRS system 330 may be controlled by a fNIRS control device, separate from the processor device 310. The processor device 310 may also control the sensor 380, if present.

In embodiments where the received data includes evoked response data, the system 300 may comprise at least one stimulator for delivering the at least one stimulus to the subject. For example, the at least one stimulus may comprise at least one auditory stimulus and/or at least one visual stimulus. The system may accordingly comprise an auditory stimulator 340 and/or a visual stimulator 350, configured to deliver the respective auditory and/or visual stimuli to the subject.

The auditory stimulator 340 and visual stimulator 350 may be directly or indirectly controlled by the processor device 310, as indicated by the dashed lines in FIG. 3. For example, the processor device 310 may include a sound stimulus output module configured to cause the auditory stimulator 340 to deliver the auditory stimulus and/or a visual stimulus output module configured to cause the visual stimulator 350 to deliver the visual stimulus. Alternatively, the auditory stimulator 340 and/or visual stimulator 350 may be controlled (together or independently) by one or more controllers distinct from the processor device 310, manually, or otherwise.

Optionally, the system 300 may further comprise a display 360. The display 360 may be configured to display the at least one classification result. In some embodiments, the display 360 (or an alternative display) may also be configured to display information related to the operation of the fNIRS system 330 and/or the sensor 380. In other embodiments, the visual stimulator 350 may be operable as a display for displaying the at least one classification result and/or information related to the operation of the fNIRS system 330. The system 300 may also comprise one or more user input modules 370 for facilitating user interaction with the system 300.

Methods for characterising tinnitus using fNIRS have been described in International Publication No WO 2022/047546, the contents of which are hereby incorporated by reference in their entirety. Methods according to the present disclosure may incorporate one or more features of the methods as described in WO 2022/047546. The present disclosure has recognized that cardiac activity data may be an indicator of physiological stress and that subjects with tinnitus may demonstrate increased sympathetic nervous activity, indicative of increased stress levels. The present disclosure has determined that inputting feature values of cortical activity data and cardiac activity data into the model may improve classification performance, such as classification accuracy between individuals with and without tinnitus, as well as classification of tinnitus at different levels of severity, compared to cortical activity data alone.

### Example 1A

Twenty-seven participants with tinnitus and twenty-one healthy adults with no history of tinnitus, neurological or hearing disorders were recruited to take part in a study. Data from one healthy participant was excluded due to technical issues. Testing was performed during a single test session.

Pure tone audiometry at frequencies of 0.25, 0.5, 1, 2, 4, and 8 kHz, was performed on all participants. Severity of tinnitus was assessed using the Tinnitus Handicap Inventory (THI). The THI is a 25-item test which quantifies the perceived severity of tinnitus on a scale of 0-100. Severity categories including slight, mild, moderate, severe and catastrophic, are associated with different scores (e.g. 0-16 slight tinnitus). Participants with tinnitus were also asked to rate the loudness and annoyance of their tinnitus on a scale of 1 to 10. The Perceived Stress Questionnaire (PSQ) was completed by all participants, except one control, to obtain a subjective rating of their stress levels. The PSQ consists of 30 questions and based on answers, provides an index between 0 and 1 with higher indices indicating higher stress. Demographic and clinical data are shown in Table 1, below.

TABLE 1
Participant demographics

| | Controls | Tinnitus | |
|---|---|---|---|
| No. of participants | 20 | 27 | |
| Gender (male:female) | 6:14 | 17:10 | |
| Age, mean (SD) | 65.9 (13.8) | 61.4 | (13.5) |
| Handedness | R: 19; L: 1 | R: 24; L: 3 | |
| THI, mean (SD) | N/A | 37.6 | (22.7) |
| Tinnitus loudness, mean (SD) | N/A | 5.3 | (2.5) |
| Tinnitus annoyance, mean (SD) | N/A | 5 | (2.9) |
| Tinnitus duration, mean (SD) | N/A | 15.2 | (12.5) |
| Tinnitus laterality | N/A | R: 2; L: 2; Both: 23 | |
| PSQ, mean (SD) | 46.8 (11.3) | 58.5 | (14.4) |

A multi-channel continuous-wave fNIRS system 500 was used to collect cortical activity data. The fNIRS system 500 had 16 light sources operating at 760 and 850 nm, and 16 detectors (NIRScout, NIRx Medical Technologies LLC). Source-detector pairs forming channels, were arranged over frontal, occipital and auditory cortical regions as shown in FIGS. 2 to 5, using NIRSite software (NIRx Medical Technologies LLC) which uses an ICBM-152 head model. MNI coordinates corresponding to channel locations were exported from NIRSite and used in AtlasViewer software to determine brain regions corresponding to the different channel locations.

The source 510 and detector 520 in most source-detector pairs were positioned 30 mm apart, forming 36 ‘long’ channels 530 (indicated in FIG. 5 by the black connecting lines between sources 510 and detectors 520). In each of the four cortical regions (frontal, left and right temporal and occipital), one ‘short’ channel 531 (indicated in FIG. 5 by asterisks) was defined by placing a source and detector 11 mm apart. The short channels 531 were configured to detect and record systemic signals from superficial layers of the subject's head (including the scalp and skull) which can interfere with detection of deeper, cortical signals. The recordings from the short channels were used to remove systemic artefacts from the fNIRS signals received from the long channels.

Data was collected using an “Empatica E4” (Empatica Inc, USA) wearable device, placed on the non-dominant wrist of all participants. The Empatica E4 was configured to record multi-modal physiological data including cardiac activity data 120 and also Electrodermal activity (EDA) data. Cardiac activity and EDA are both known to be associated with physiological stress responses. EDA data was captured using silver plated electrodes to measure changes in electrical conductance across the skin as a result of changes in sweat gland activity with different emotional states. Cardiac activity data 120 in the form of blood volume pulse measurements was captured using a photoplethysmography sensor. Heart rate (HR) and inter-beat interval data were derived from the blood volume pulse measurements and exported from Empatica. Empatica data was synchronised with the simultaneously recorded fNIRS data using the common system time on the recording computer. The Empatica E4 records multimodal physiological data including EDA using silver plated electrodes at 4 Hz, and blood volume pulse using a photo-plethysmography (PPG) sensor at 64 Hz. HR data is calculated from PPG using a proprietary algorithm and exported at 1 Hz (one sample per second). Inter-beat intervals were exported as time and duration of the detected inter-beat intervals. Measures of heart rate variability (HRV) were derived from the inter-beat intervals.

A plurality of discrete auditory stimuli were delivered binaurally to each subject via audiometric insert earphones (ER-3A insert earphone, E-A-RTONE™ 165 GOLD, USA) and calibrated using a Norsonic sound level meter (Norsonic AS, Norway). Each auditory stimulus consisted of a 15-second segment of pink noise delivered at 65 dB Sound Pressure Level (SPL).

A plurality of visual stimuli were delivered to each subject as a reversing display of circular checkerboard patterns, with pattern reversal at a temporal frequency of 7.5 Hz (15 reversals per second). This pattern produces strong cortical responses in people with good visual acuity. The images were radial in nature and consisted of rings, divided into sectors with neighbouring sectors of opposite colour (black and white).

Each test session was performed as discussed above in relation to FIG. 3. Testing was performed in a sound-treated booth with subjects sitting on a comfortable chair and auditory and visual stimuli presented using Presentation Software (Neurobehavioral Systems, USA).

Pre-processing of the fNIRS signals was performed in Matlab 2022a (Mathworks, USA), the NIRS Brain AnalyzlR Toolbox and custom-written Matlab scripts. Resting-state and evoked response fNIRS signals were down-sampled to 1 Hz and 3 Hz, respectively and converted to optical density. Short channel correction was applied to remove extracerebral signals (such as fluctuations from scalp and systemic responses and respiration) from long channels. Correction was performed by subtracting a scaled version of the nearest short channel from each long channel. For evoked response recordings, signals were band-pass filtered between 0.01-0.12 Hz by applying zero-phase 8th order Butterworth high-pass (at 0.01 Hz) and low-pass (0.12 Hz) filters respectively. Oxygenated and de-oxygenated haemoglobin (HbO and HbR) were then estimated using the modified Beer-Lambert law.

Channels with poor signal quality were identified using two methods. Channels with gains over 7 showing optical signals within the range 0.09-1.4 V and noise levels less than 2.5%, were marked as having inadequate detected light intensity. In addition, channels with poor cardiac signal content as quantified using low correlation (<0.75) between detected signals at 760 and 850 nm filtered between 0.2 to 2.5 Hz were marked as having poor signal quality. Channels with poor signal quality were excluded from analysis.

Feature values were extracted from HbO and HbR signals recorded at rest and in response to auditory and visual stimuli, as described below.

### Resting-State Cortical Activity Data

A seed analysis method was used to determine a resting-state functional connectivity measure from the resting-state cortical activity data. Single fNIRS channels over the left and right superior temporal gyrus were selected as seed channels and correlations between these seed channels and other channels were calculated using whitened correlations. Each correlation value was used as a feature value. This correlation method may avoid false correlations due to non-normal noise structures introduced by the slow haemodynamic signals and artefacts.

### Evoked Cortical Activity Data

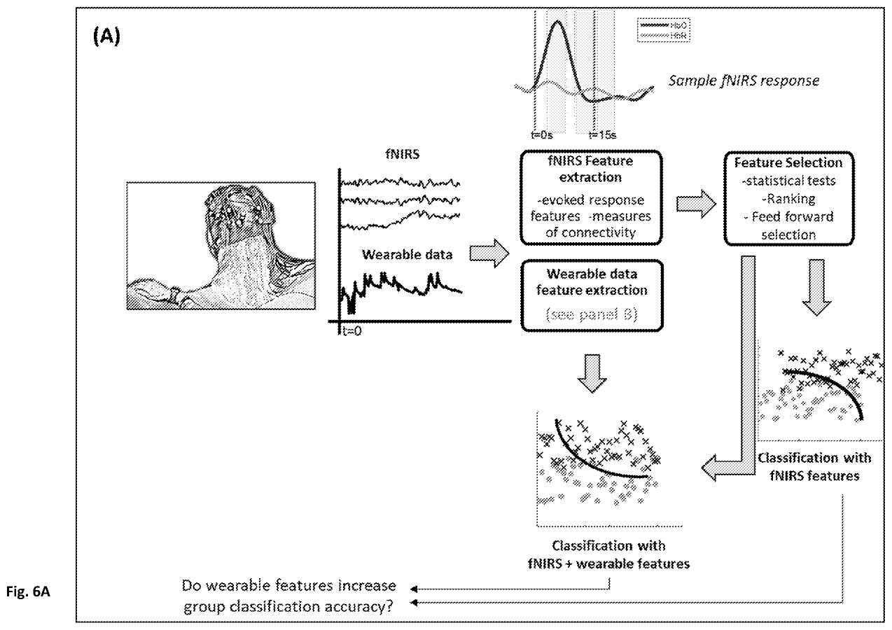

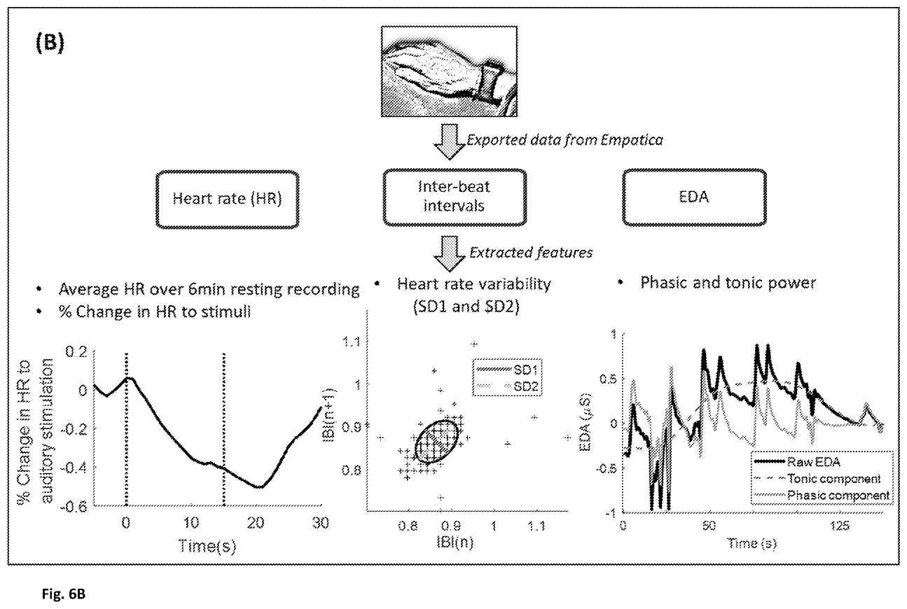

Averaged amplitudes of the evoked cortical activity data fNIRS signals over three different time-windows were used as feature values. Time windows of 3-8 seconds, 10-15 seconds and 15-20 seconds, relative to the start of applying of an auditory or visual stimulus, were chosen based on onset and offset of stimulus times and response peak times observed in grand averaged responses in our current and previous studies. A sample response to auditory stimulation with time-windows highlighted, is shown in the uppermost graph in FIG. 6A.

### Cardiac Activity Data

Average resting-state heart rate was calculated for each subject based on the resting-state cardiac activity data. Two measures of heart rate variability (HRV), indicating short term and long-term heart rate variability, respectively, were also calculated from the recorded cardiac activity data using Poincaré plots. A Poincaré plot is obtained by plotting a sequence of intervals between consecutive heartbeats. Each interval is plotted against the following interval, displaying the correlation between intervals. One method for quantifying HRV is fitting an ellipse to the datapoints and measuring the dispersion of the datapoints within the ellipse along either the major axis (SD1) or the minor axis (SD2) of the ellipse, as shown in FIG. 6B, indicating short-term and long-term variability of heartbeats, respectively.

A percentage change in heart rate in response to both auditory and visual stimuli, was calculated based on the evoked cardiac activity data. The change for each epoch was calculated relative to baseline, defined as heart rate averaged over five seconds prior to the stimulus onset. The percentage change in heart rate over the stimulus time-window (0-15 s) was used as a feature value.

### Electrodermal Activity Data

Skin conductance was measured using the tonic and phasic components of the acquired EDA signal. These components measure the underlying slow (tonic) and rapid (phasic) changes of the signal, in response to stimuli. To extract the tonic and phasic components of the recorded signal, the EDA time series data was first normalised corresponding to each stimulus with respect to the minimum and maximum signal amplitude (z-score) in the preceding 6-minute rest period of the same session. For each stimulus, the signal was then filtered using a Butterworth bandpass filter below 0.02 Hz to obtain slow varying or tonic component and above 0.02 Hz to obtain the rapidly varying or phasic component. The absolute signal power of each of these components, summed over the entire time series representing each stimulus was then calculated.

### Feature Extraction

Features extracted from the cortical activity data (fNIRS signals), cardiac activity data and electro-dermal activity data signals were grouped into the following classes:
Controls versus tinnitus; and
Within the tinnitus group:
   Slight to mild versus moderate, severe or catastrophic tinnitus (based on THI categories);
   Low (<6 on the visual rating scale) versus high loudness (>=6 on visual rating scale); and
   Low (<6 on the visual rating scale) versus high annoyance (>=6 on visual rating scale).

For each group, the following feature selection method was applied. Group comparisons were performed using the Kruskal-Wallis test to identify features which showed significant group differences (p<=0.05). The Kruskal-Wallis test was used as a non-parametric test as some features showed non-normal distributions. Identified feature values were then ranked based on the p-values obtained. Lower p-values indicate a greater difference between groups and therefore resulted in a higher ranking for the associated feature values.

A feed-forward feature selection method was then used to select a subset of features from those identified above. The highest ranked feature was used to classify the groups with classifier performance evaluated using 5-fold cross-validation accuracy. Subsequent features were then added one at a time and classification repeated. A feature value was retained if classification accuracy was equal to or higher than the previous iteration, and removed if accuracy of the model decreased.

### Classification

The selected features were then used with machine learning methods including feature selection and classifiers, to classify subjects as controls or experiencing tinnitus and/or to differentiate the subjects based on tinnitus severity. The subjects were classified as having slight/mild versus moderate/severe tinnitus (based on THI ratings).

Features input to the machine learning algorithms included features from the resting-state cortical activity data and evoked cortical activity data described above. Classification models were trained using the selected features. Classifiers assessed included K-nearest neighbour (KNN, with cosine and Euclidean distance, K=10), support vector machine (SVM, with both linear and gaussian kernels) and decision trees. The choice of classification models was based on both complexity and interpretability. KNN provided a simple supervised method to classify data based on the distance of the test sample from each class of available training samples, and was implemented with two different distance measures, i.e., Euclidean distance and cosine distance. Further, SVMs with different kernel types (linear and gaussian) were trained to classify data based on the maximal-margin hyperplane concept. Finally, decision trees (split criterion: Gini's diversity index) and their bootstrap aggregation or bagged ensembles, with random forest as the ensemble aggregation method, were used for rule-based classification from feature values. Classifier performance was calculated using 5-fold cross-validation and the average sensitivity (true positive rate), specificity (true negative rate), accuracy (number of correctly predicted samples over total number of samples) and F1-score averaged over the 5 folds. F1-score is also a measure of accuracy that may be better suited to imbalanced data in different groups.

Classification was then repeated with addition of features from the cardiac activity data to assess whether classification accuracy was improved.

### Tinnitus Vs Controls

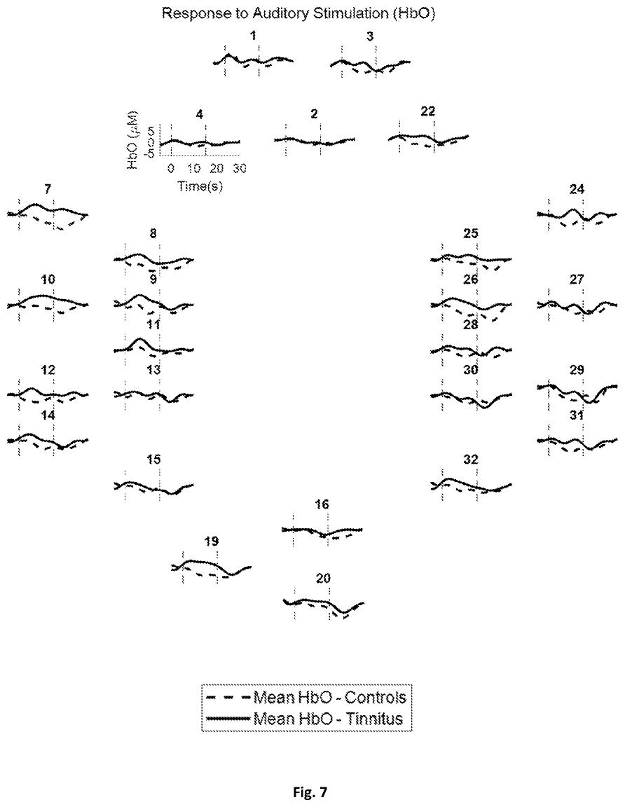

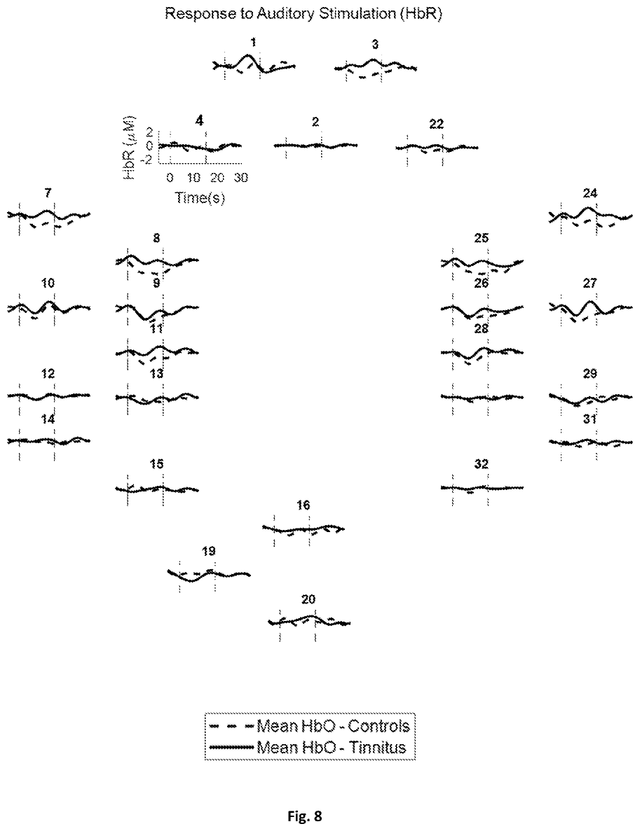

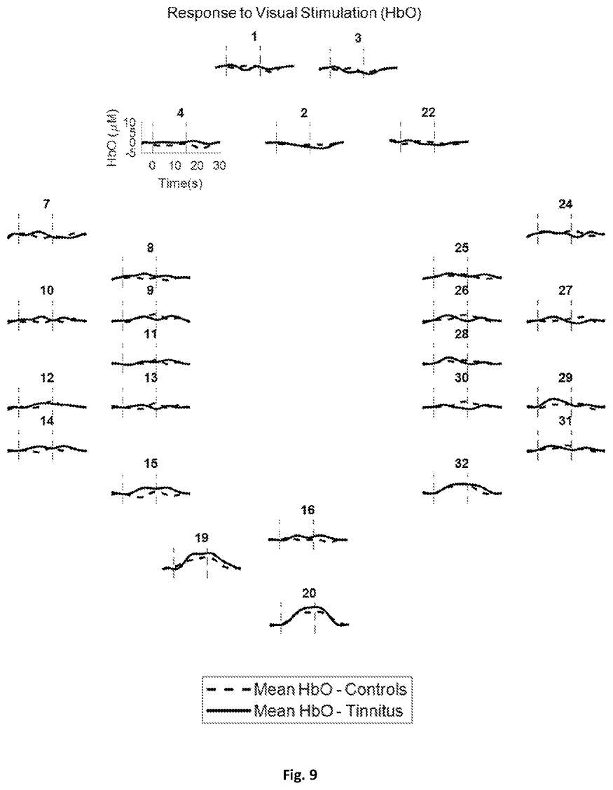

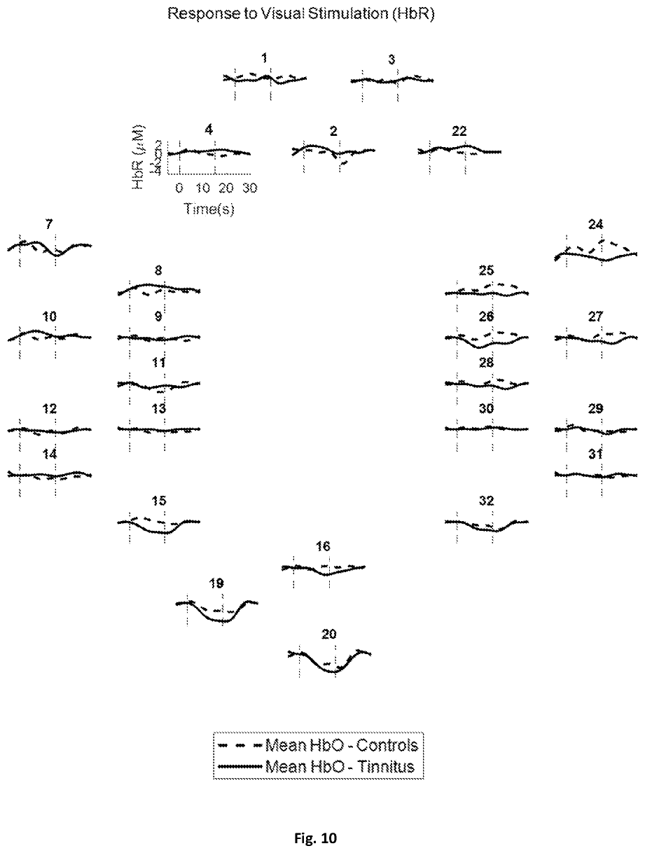

Group averaged HbO and HbR evoked cortical activity fNIRS signals for tinnitus participants and controls in response to auditory or visual stimuli are shown in FIGS. 7 to 10. To account for cross-modal activity, evoked cortical activity data in response to auditory stimuli are shown in channels in both auditory and non-auditory (frontal and visual) regions. Likewise, evoked cortical activity data in response to visual stimuli are shown in both visual and non-visual (frontal and auditory) regions. Group averaged measures of connectivity between left and right seeds with all channels, are shown in FIGS. 11A and 11B, respectively. As described above, the feature selection process involved identifying features which showed significant group differences, ranking these features, and selecting a subset of the features which increased classification accuracy.

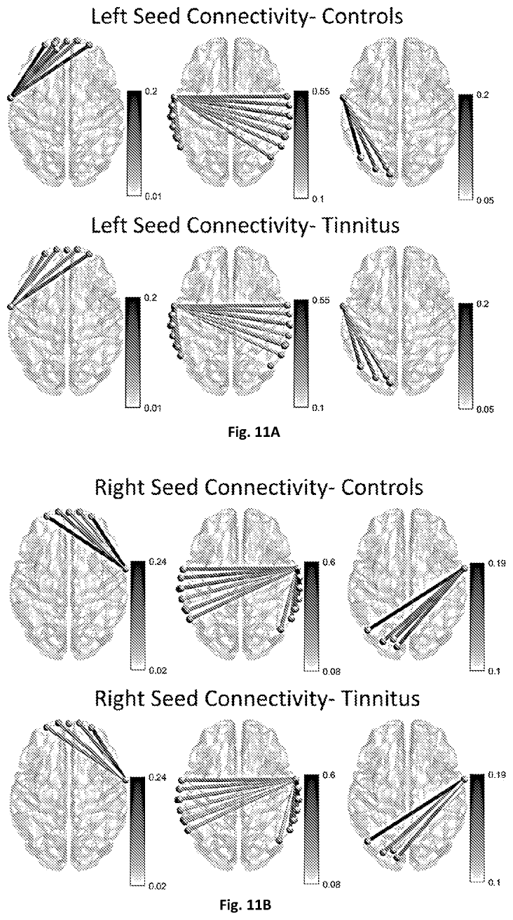

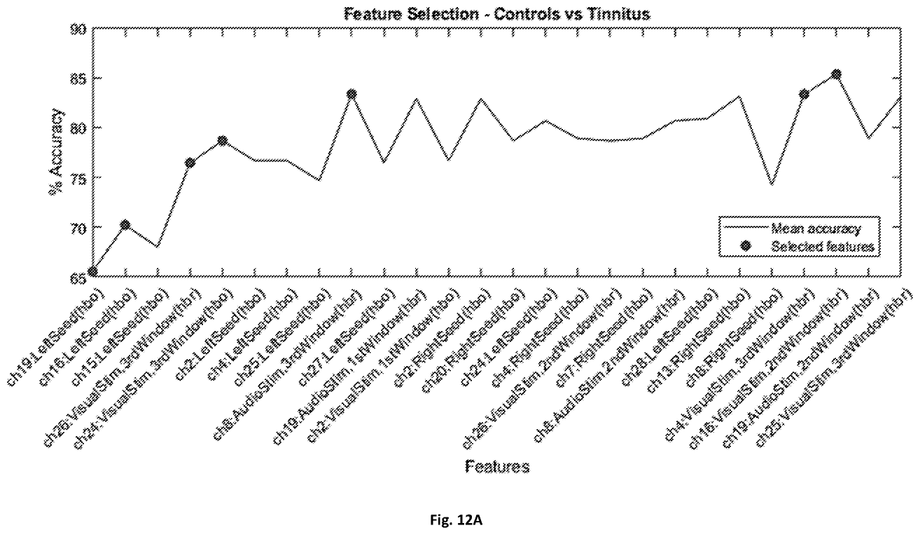

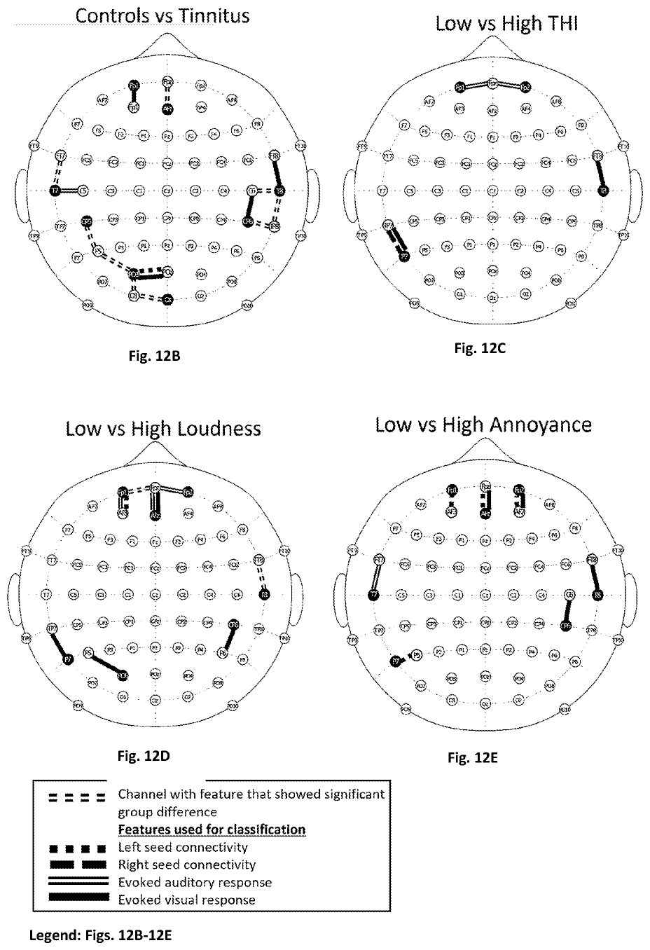

FIG. 12 illustrates results of feature ranking using a linear SVM classifier. When comparing tinnitus to controls, 15 connectivity features and 11 evoked response features showed significant group difference between tinnitus and controls. As shown in FIG. 12A of these features, two connectivity and four visual response features and one auditory, were found to increase group classification accuracy. These features were included in the selected feature set. The selected features included HbO-derived left seed connectivity with channels in the frontal, and visual regions. Both connectivity measures were higher in the control group when compared to tinnitus. Selected features also included HbR visual responses recorded from one channel in the frontal region, two channels in the right auditory region and one channel in the visual region. A HbO auditory response feature in one channel in the left auditory region was also selected.

### Within Tinnitus Groups

Data from tinnitus participants were categorised into different groups based on THI categories, loudness ratings and annoyance ratings.

Data from tinnitus participants were categorised into two groups based on THI categories: slight or mild tinnitus (low THI, n=13) versus moderate, severe or catastrophic tinnitus (high THI, n=14). As shown in FIG. 12C, a total of seven features of the evoked cortical activity data and one resting-state connectivity feature showed significant group differences. Of these features, six features of the evoked cortical activity data and one connectivity feature were selected using the forward-selection algorithm. Selected features included HbR auditory response features in 1 channel in the frontal region and 3 HbO visual response features in two channels in the auditory regions and HbR-derived right seed connectivity with a channel in the left auditory region.

Tinnitus participants who rated their loudness as less than six (i.e. low loudness, n=14) were compared to those with loudness ratings above or equal to six (high loudness, n=13). As shown in FIG. 12D, a total of 12 features of the evoked cortical activity data and two features of the resting-state functional connectivity measure showed significant group differences. Features selected using the forward-selection algorithm included eight features of the evoked cortical response data (HbO and HbR auditory responses in frontal channels and visual response feature in frontal, left and right auditory region) and one feature of the resting-state connectivity measure, with a frontal channel. Absolute connectivity values were higher in the high loudness group.

Tinnitus participants with annoyance ratings less than 6 (low annoyance, n=16) were compared to those who rated their annoyance levels greater than or equal to 6 (high annoyance, n=11). In total, 6 features of the evoked cortical activity data and four features of the resting-state functional connectivity measure showed significant group differences. From these features, five features of the evoked cortical activity data and four features of the resting-state functional connectivity measure were selected. Features of the evoked cortical activity data included auditory response in a left auditory channel and visual responses in frontal and right auditory channels. Features of the resting-state functional connectivity measure included left seed to frontal and left auditory channel features. Absolute connectivity values were higher in the high annoyance group.

Feature selection was initially performed on cortical activity data features alone, with the following findings: left seed connectivity to channels in frontal and occipital channels, and auditory response feature in the left auditory region, and visual response features in the frontal, right auditory and visual region, contributed to classification of tinnitus subjects from controls.

Within the tinnitus group, features selected for classification of different tinnitus groups were from frontal and auditory channels. Features of the evoked cortical activity data in response to auditory stimulus in frontal channels were among features selected for classification of low versus high THI and loudness groups. A feature of the evoked cortical activity data in response to a visual stimulus in a frontal channel contributed to classification of low versus high tinnitus loudness and annoyance groups. Left and right seed resting-state functional connectivity measures with frontal channels contributed to distinguishing both low versus high loudness and annoyance groups but not low versus high THI groups.

Features from channels in visual regions of interest did not contribute to classification of any of the tinnitus groups. However visual response features in the left and right auditory regions contributed to classification of the different tinnitus groups.

Cardiac Activity/EDA Features with PSQ and Annoyance Ratings

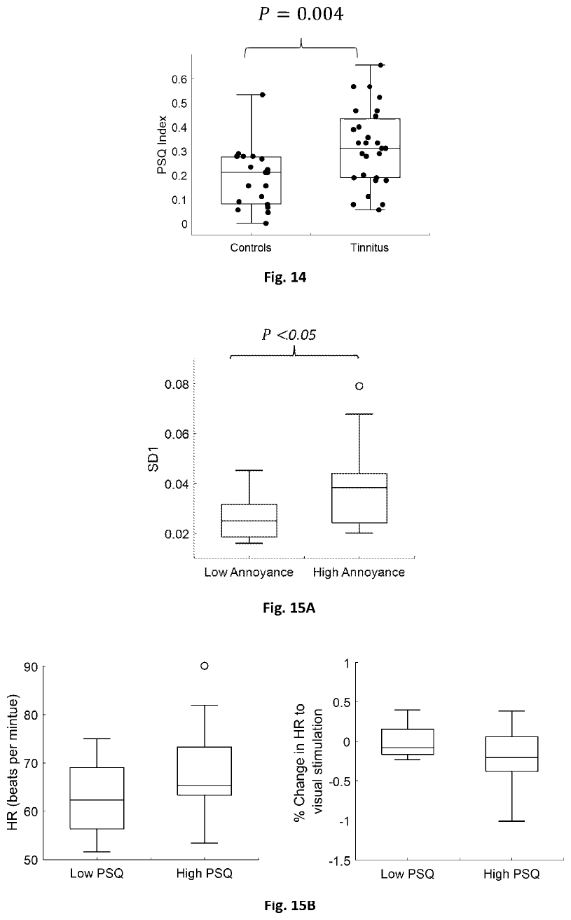

FIG. 14 shows that stress levels, as rated by PSQ, were significantly higher in the tinnitus group when compared to controls. Individuals with tinnitus were grouped into a low (PSQ<0.3, n=12) versus high (PSQ>0.3, n=15) PSQ group. Within the tinnitus group, annoyance ratings and PSQ scores were not significantly correlated (ρ=0.3, p=0.11), and therefore were both used to assess whether different PSQ and annoyance ranges were reflected by cardiac activity-related or EDA measures. Mean resting HR, percentage change in HR to stimulation, HRV (SD1, SD2) as well as tonic and phasic EDA were compared in tinnitus participants with low versus high annoyance as well as low versus high PSQ ratings.

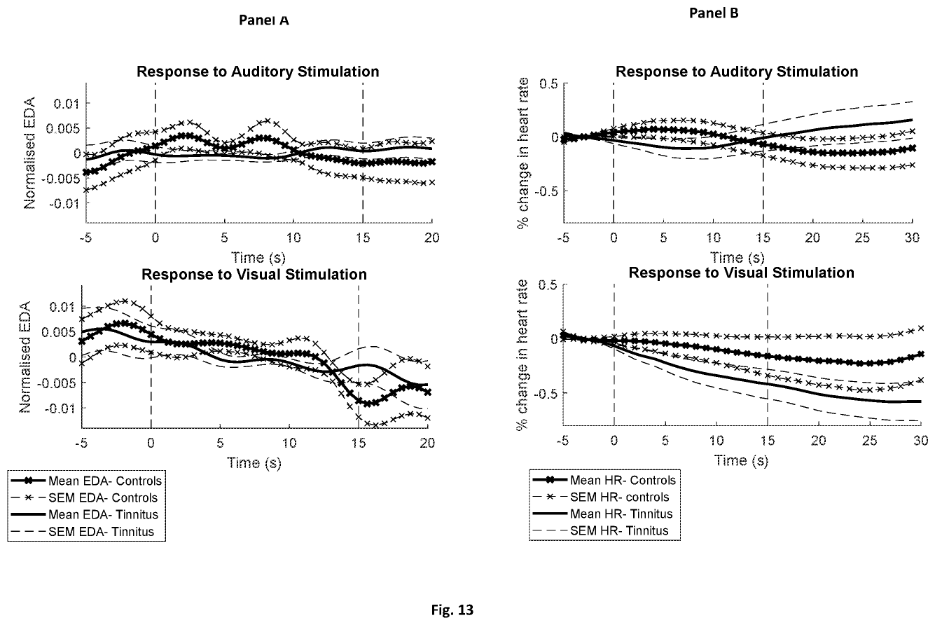

As shown in Panel A of FIG. 13, despite EDA measures, no significant group differences (PSQ or annoyance groups) were found. FIG. 15A shows significantly higher HRV SD1 values in the high annoyance group compared to the low annoyance group. FIG. 15B shows a comparison of low versus high PSQ groups, which did not show significant differences in the HR related measures. However, resting HR and change in HR in response to visual stimulation approached significance. Trends observed were higher group averaged resting heart rate and a bigger reduction in heart rate with visual stimulation in the high PSQ group compared to the low PSQ group.

Based on the changes observed in cardiac activity data-derived features with different PSQ or annoyance groups, these features were added to the classifiers, as described below. As EDA measures did not show associations with PSQ or annoyance measures, EDA features were not included.

### Classification

Tables 2-4 below set out classification performance.

Table 2 shows classification performance obtained with features of cortical activity data alone. The highest F1-scores (also considering high sensitivity and specificity) obtained for the various groups were as follows. The highest score for differentiating tinnitus from controls was 0.85 using a cosine KNN classifier with average sensitivity and specificity of 0.80 and 0.89 across 5-folds.

Within the tinnitus group, the highest score when classifying low versus high THI categories (Table 3) was 0.89 obtained using bagged trees. When classifying tinnitus subjects with low versus high loudness ratings (Table 3) an F1-score of 0.88 was achieved using a cosine KNN classifier. An F1-score of 0.89 was achieved using a Euclidian KNN to classify tinnitus subjects with low versus high annoyance ratings (Table 3).

Table 4 shows results from combining features from cardiac activity data with features from cortical activity data and using the classifiers that performed best with fNIRS cortical activity data alone. The addition of cardiac activity data improved performance for linear and Gaussian SVM classifiers, when differentiating tinnitus subjects with low versus high loudness ratings. Smaller improvements were seen with two classifiers for low/high THI groups.

TABLE 2
Classification outcomes (f1-scores) for differentiating patients
with tinnitus from controls using fNIRS features alone.

| Control vs | Sensitivity | | Specificity | | Accuracy | | f1-score | |
|---|---|---|---|---|---|---|---|---|
| Tinnitus | M | SD | M | SD | M | SD | M | SD |
| Euclidian KNN | 0.81 | 0.19 | 0.84 | 0.23 | 0.79 | 0.10 | 0.80 | 0.08 |
| Cosine KNN | 0.80 | 0.13 | 0.89 | 0.14 | 0.85 | 0.05 | 0.85 | 0.07 |
| Linear SVM | 0.82 | 0.09 | 0.84 | 0.13 | 0.83 | 0.08 | 0.85 | 0.06 |
| Gaussian SVM | 0.87 | 0.13 | 0.63 | 0.21 | 0.74 | 0.11 | 0.79 | 0.09 |
| Coarse Tree | 0.57 | 0.15 | 0.60 | 0.36 | 0.64 | 0.05 | 0.62 | 0.11 |
| Bagged Tree | 0.73 | 0.25 | 0.65 | 0.35 | 0.72 | 0.11 | 0.71 | 0.17 |

TABLE 3
Classification outcomes (f1-scores) for differentiating
different groups within patients with tinnitus: slight
or mild tinnitus (low THI) versus moderate, severe or
catastrophic tinnitus (high THI); low (<6) versus high
(>=6) loudness and low (<6) versus high (>=6) annoyance.

| Tinnitus | Sensitivity | | Specificity | | Accuracy | | f1-score | |
|---|---|---|---|---|---|---|---|---|
| Group | M | SD | M | SD | M | SD | M | SD |
| Mild vs Severe | | | | | | | | |
| THI | | | | | | | | |
| Euclidian KNN | 0.57 | 0.39 | 0.58 | 0.38 | 0.48 | 0.15 | 0.45 | 0.25 |
| Cosine KNN | 0.95 | 0.10 | 0.80 | 0.27 | 0.85 | 0.15 | 0.86 | 0.13 |
| Linear SVM | 0.95 | 0.10 | 0.83 | 0.21 | 0.85 | 0.13 | 0.86 | 0.13 |
| Gaussian SVM | 0.87 | 0.16 | 0.48 | 0.26 | 0.69 | 0.17 | 0.75 | 0.11 |
| Coarse Tree | 0.83 | 0.21 | 0.63 | 0.37 | 0.73 | 0.21 | 0.75 | 0.18 |
| Bagged Tree | 0.96 | 0.08 | 0.85 | 0.20 | 0.89 | 0.09 | 0.89 | 0.12 |
| Low vs High | | | | | | | | |
| Loudness | | | | | | | | |
| Euclidian KNN | 0.65 | 0.37 | 0.67 | 0.37 | 0.63 | 0.22 | 0.58 | 0.32 |
| Cosine KNN | 0.87 | 0.16 | 0.93 | 0.13 | 0.89 | 0.09 | 0.88 | 0.10 |
| Linear SVM | 0.78 | 0.30 | 0.88 | 0.15 | 0.79 | 0.16 | 0.73 | 0.20 |
| Gaussian SVM | 0.57 | 0.23 | 0.83 | 0.21 | 0.67 | 0.17 | 0.61 | 0.21 |
| Coarse Tree | 0.70 | 0.19 | 0.86 | 0.20 | 0.78 | 0.06 | 0.72 | 0.08 |
| Bagged Tree | 0.60 | 0.08 | 0.75 | 0.22 | 0.70 | 0.10 | 0.66 | 0.10 |
| Low vs High | | | | | | | | |
| Annoyance | | | | | | | | |
| Euclidian KNN | 0.83 | 0.21 | 1.00 | 0.00 | 0.93 | 0.09 | 0.89 | 0.14 |
| Cosine KNN | 0.83 | 0.21 | 0.93 | 0.13 | 0.85 | 0.13 | 0.85 | 0.13 |
| Linear SVM | 0.88 | 0.15 | 0.95 | 0.10 | 0.89 | 0.09 | 0.86 | 0.13 |
| Gaussian SVM | 0.67 | 0.42 | 1.00 | 0.00 | 0.81 | 0.24 | 0.70 | 0.40 |
| Coarse Tree | 0.70 | 0.24 | 0.78 | 0.30 | 0.75 | 0.18 | 0.68 | 0.17 |
| Bagged Tree | 0.87 | 0.27 | 0.87 | 0.27 | 0.84 | 0.20 | 0.83 | 0.21 |

TABLE 4
Classification outcomes (f1-scores) for different groups and using
fNIRS and heart rate-related features. Results are presented for the
three classifiers that performed best for the different groups (based
on Tables 2 and Supplementary Table) with fNIRS features alone. F1-
scores which increased with addition of HR features are indicated in
bold. For low versus high loudness groups, f1-scores increased with
addition of heart rate-related features, for two classifiers.

| | | | | | Low vs | | Low vs | |
|---|---|---|---|---|---|---|---|---|
| | Controls vs | | Low vs | | High | | High | |
| | Tinnitus | | High THI | | Loudness | | Annoyance | |
| | M | SD | M | SD | M | SD | M | SD |
| Cosine KNN | | | | | | | | |
| fNIRS features | 0.85 | 0.07 | 0.86 | 0.13 | 0.88 | 0.10 | 0.85 | 0.13 |
| fNIRS + HR | 0.86 | 0.04 | 0.88 | 0.10 | 0.86 | 0.13 | 0.77 | 0.14 |
| features | | | | | | | | |
| Linear SVM | | | | | | | | |
| fNIRS features | 0.85 | 0.06 | 0.86 | 0.13 | 0.73 | 0.20 | 0.86 | 0.13 |
| fNIRS + HR | 0.85 | 0.06 | 0.94 | 0.07 | 0.86 | 0.13 | 0.82 | 0.11 |
| features | | | | | | | | |
| Gaussian SVM | | | | | | | | |
| fNIRS features | 0.79 | 0.09 | 0.75 | 0.11 | 0.61 | 0.21 | 0.70 | 0.40 |
| fNIRS + HR | 0.79 | 0.06 | 0.58 | 0.17 | 0.73 | 0.37 | 0.87 | 0.16 |
| features | | | | | | | | |

Based on these findings, combining features from cardiac activity data 120 with features from cortical activity data 110, from resting-state and/or evoked response data recordings, may improve classification of subjects with tinnitus, at least at low versus high THI and loudness levels with a larger increase seen for loudness levels.

### Example 1B

In further testing, using the protocols described above for Example 1A, fNIRS signals in response to auditory or visual stimuli were also recorded from a larger sample of tinnitus participants and controls (41 controls and 90 individuals with diagnosed tinnitus). There was no significant difference in age (mean (SD) age: controls 66.1 (7.6); tinnitus 63.7 (7.3) years; p=0.8) or gender (λ(1)=3.8, p=0.05) between participants.

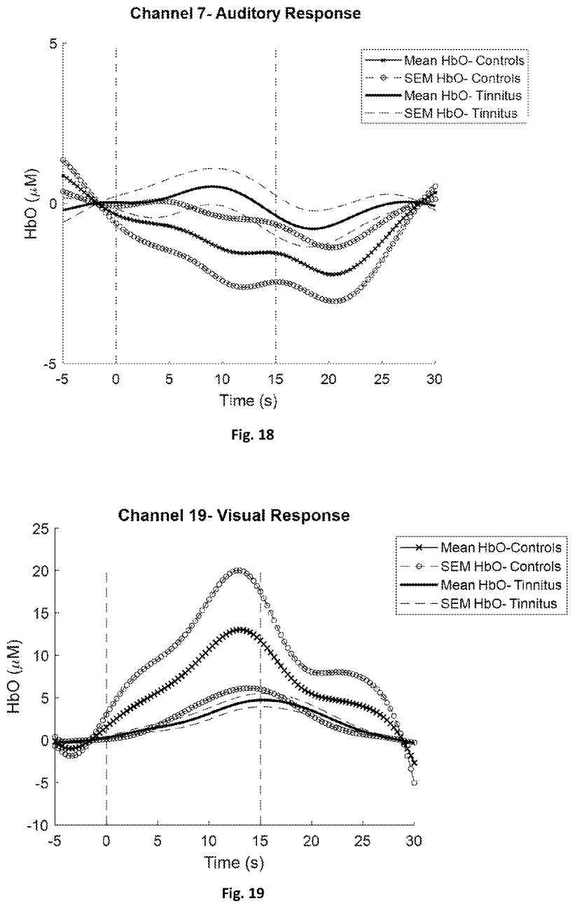

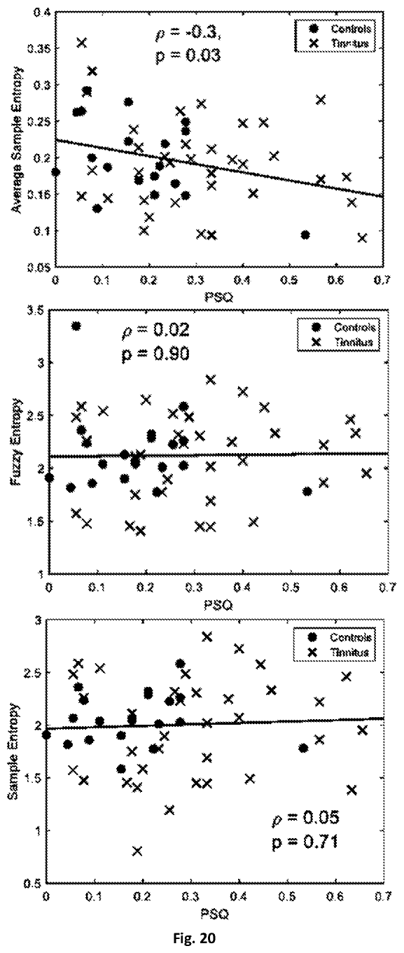

Group averaged HbO and HbR responses to auditory and visual stimuli were calculated. Group averaged auditory responses from a channel in the superior temporal gyrus are shown in FIG. 18. Group averaged visual responses from a channel in the occipital region are shown in FIG. 19. Amplitude measures were extracted from auditory and visual responses and were used to train classifiers as described below. Feature values including heart rate and/or heart rate variability were also used, as described below.

### Example Classifier Training Method:

1. 10% of dataset was separated.
2. With the remaining 90% of the dataset, a subset (80%) was used to determine features of cortical activity data and features of cardiac activity data that showed significant group differences.
3. Step 2 was iterated 30 times, with a random subset (80%) used each time.
4. Feature values which showed significant group differences more than 10 times (out of the 30 iterations) were used to train classifiers twice: with features of cortical activity data alone and with features of cortical activity data combined with features of cardiac activity data.
5. Performance of the models was evaluated using the 10% of the dataset set aside in step 1.
6. Accuracy of classification when using features of cortical activity data alone was compared to accuracy of classification when using features of cortical activity data with features of heart rate and heart rate variability.

The accuracy of detecting a presence or absence of tinnitus in the subject (in this case, distinguishing between controls and tinnitus patient data) using a tri-layered artificial neural network (ANN) increased from 61% when using features of cortical activity data alone to 76% when using features of cortical activity data and features of cardiac activity data in combination.

Using features of cardiac activity data (heart rate and heart rate variability) alone in the absence of features from cortical activity data, classifying controls versus tinnitus had an accuracy of 54%. These results indicate that inputting features values of cortical activity data in combination with feature values of cardiac activity data to the model provides improved performance when classifying a presence or absence of tinnitus (in this instance, differentiating subjects with diagnosed tinnitus from controls), compared to use of feature values of either cardiac or cortical activity data alone.

### Example 2A

This study uses one method of HRV analysis, called entropy profiling, to objectively assess tinnitus related stress in subjects.

Physiological models of tinnitus and subjective patient reports relate tinnitus to stress. Measures of heart rate variability (HRV) may be used as a non-invasive measure, indicative of a stress level in a subject. However, most methods of characterising HRV require relatively long data recordings.

Entropy measures quantify signal regularity and are commonly used in HRV analysis. One factor in the accuracy of HRV measures is the length and quality of the underlying cardiac recordings. At least 20 minutes of data is recommended for accurate results when using conventional entropy measures. However, obtaining long recordings is challenging in clinical settings. Although longer recordings are likely to contain more information, they are also prone to movement artefacts which introduce noise in the signals.

A recently developed technique for assessing HRV is called “entropy profiling”. Entropy profiling has been shown to provide high accuracy in identifying changes in HRV due to cardiac arrhythmia as well as due to ageing. Unlike other entropy measures which require long periods of data, entropy profiling can extract information from short-length data recordings of only 2-15 minutes.

Other commonly used entropy measures include Sample Entropy and Fuzzy Entropy. These methods are limited by the need for appropriate choice of a tolerance parameter, r, for their calculation. Inappropriate choice of this parameter can adversely affect the reliability of these measures. Entropy profiling addresses this issue by automatically selecting an appropriate range and resolution for the parameter r, based on the input time-series. This data-driven, non-parametric approach estimates a set of r values, from which a complete approximate entropy profile is generated.

This study assesses the relationship between measures of entropy derived from cardiac activity data recordings, with subjective ratings of stress in individuals with tinnitus. The performance of entropy profiling, and its correlation with subjective stress ratings, is compared to that of conventional methods of entropy calculation including sample entropy and fuzzy entropy.

Cardiac activity data was collected using a wearable wristband from 15 individuals with tinnitus and 17 controls. There was no significant difference in age (mean (SD) age: controls 69.8 (6.7); tinnitus 69 (5.5) years; t(28)=0.0, p=0.7) or gender (λ(1)=3.03, p=0.08) between participants. Testing was done during a single session.

Severity of tinnitus was assessed using the Tinnitus Handicap Inventory, a 25-item test which rates the perceived severity of an individual's tinnitus on a scale of 0-100. The Perceived Stress Questionnaire (PSQ) was completed by all participants to obtain a subjective rating of their stress levels.

An Empatica E4 wristband (Empatica Inc, USA) was placed on the non-dominant wrist of all participants. Resting-state cardiac activity data was collected over a period of 6 minutes, in which subjects sat still with their eyes closed. The Empatica E4 records physiological data including blood volume pulse using a photoplethysmography sensor. Inter-beat intervals (IBIs) were derived from blood volume pulse and exported from Empatica as time and duration of the detected inter-beat intervals. For each participant, IBI values outside+2SD of the total IBI values over six minutes, were rejected. For the resulting IBI values, entropy features were calculated.

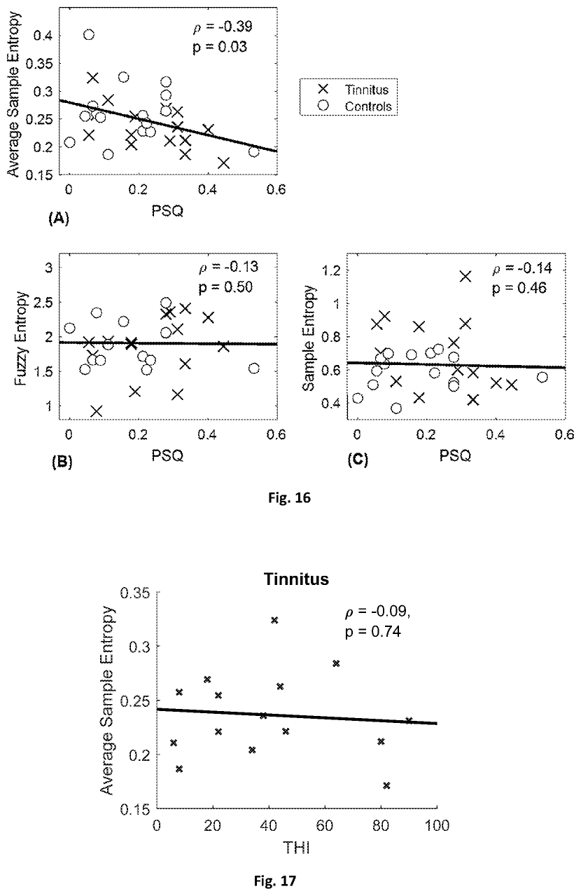

FIG. 16 shows entropy measures, as calculated using different methods, versus PSQ subjective rating scores. As seen in FIG. 16, Panel A, the entropy profiling method (which yields the “average sample entropy” measure) shows significant correlation with PSQ ratings of stress across data from both tinnitus participants and controls. A higher correlation is found for data from tinnitus participants only (ρ=−0.57, p=0.03). FIG. 16 Panel B and Panel C show no significant correlation between fuzzy entropy and sample entropy and PSQ scores. This finding was seen with data from both tinnitus and control subjects included as well as with data from tinnitus subjects alone. This may be due to the choice of r=0.15*SD not being suitable for the short data lengths in this study. This highlights the potential suitability of the entropy profiling method over fuzzy entropy or sample entropy for use with data recordings of relatively short lengths, such as those produced in embodiments of the methods according to the present disclosure.

The use of short-term data may enable a more clinically applicable approach to objective assessment of stress in tinnitus, which may be useful in monitoring tinnitus and/or assessing efficacy of relevant interventions or treatments for this condition.

As shown in FIG. 17, no significant correlation between average sample entropy and tinnitus handicap inventory (THI) scores was found, suggesting that stress severity may be monitored independently from tinnitus severity.

### Example 2B

In further testing, using the methods described above for Example 2A, fNIRS recordings were obtained from subjects at rest, on a larger dataset of 41 controls and 90 individuals with diagnosed tinnitus. There was no significant difference in age (mean (SD) age: controls 66.1 (7.6); tinnitus 63.7 (7.3) years; p=0.8) or gender (λ(1)=3.8, p=0.05) between participants. In this testing, cardiac activity data was derived from the recorded fNIRS signals, rather than from a separate sensor. Entropy measures were calculated from heart-rate data, using the methods as described above in relation to FIG. 16. As shown in FIG. 20, average sample entropy (derived using the entropy profiling method) showed a significant correlation with PSQ ratings of stress across data from both tinnitus participants and controls. Fuzzy and Sample entropy measures did not show a significant correlation with PSQ ratings.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method for characterising tinnitus in a subject, the method comprising:
receiving data at a processor device, the received data comprising:
cortical activity data, indicative of cortical activity in one or more regions of the subject's brain, the cortical activity data derived from one or more functional near-infrared spectroscopy (fNIRS) signals; and
cardiac activity data, indicative of cardiac activity in the subject;
processing the received data using the processor device, the processing comprising:
inputting, into a model, feature values including one or more features of the cortical activity data and one or more features of the cardiac activity data,
wherein the model is configured to provide one or more classification results based on the feature values, the one or more classification results being indicative of at least one characteristic of tinnitus in the subject.

2. The method of claim 1, wherein the cardiac activity data is derived from one or more signals provided by one or more electrical sensors and/or one or more optical sensors.

3. The method of claim 1, wherein the cardiac activity data is derived from the one or more fNIRS signals.

4. The method of claim 1, wherein processing the received data comprises determining heart rate data based on the cardiac activity data and wherein the feature values include one or more features of the heart rate data.

5. The method of claim 1, wherein processing the received data comprises determining heart rate variability data based on the cardiac activity data and wherein the feature values include one or more features of the heart rate variability data.

6. The method of claim 1, wherein the classification results include at least one of:
a presence or absence of tinnitus in the subject; or
a severity rating of tinnitus in the subject.

7. (canceled)

8. The method of claim 1, wherein the model provides at least one of:
quantification of loudness of the tinnitus; or
quantification of annoyance produced by the tinnitus.

9. (canceled)

10. The method of claim 1, wherein the model comprises a trained model, trained with an artificial intelligence (AI) algorithm.

11. The method of claim 1, wherein the received data comprises resting-state data comprising resting-state cortical activity data and resting-state cardiac activity data obtained while the subject is at rest.

12. The method of claim 11, wherein the resting-state data comprises at least one of:
data obtained over a period of about 6 minutes; or
data obtained prior to delivery of a stimulus to the subject.

13. (canceled)

14. The method of claim 11, wherein the resting-state cortical activity data is derived from fNIRS signals indicative of cortical activity in two or more regions of the subject's brain obtained while the subject is at rest and wherein processing the data comprises determining at least one resting-state functional connectivity measure between the at least two regions of the subject's brain, based on the resting-state cortical activity data, and wherein the feature values include one or more features of the at least one resting-state functional connectivity measure.

15. The method of claim 11, wherein processing the data comprises determining a heart rate variability and/or an average heart rate, based on the resting-state cardiac activity data, wherein the feature values include the average heart rate and/or the heart rate variability.

16. The method of claim 1, wherein the received data comprises evoked response data comprising evoked cortical activity data and evoked cardiac activity data obtained during and/or following delivery of at least one stimulus to the subject, and wherein the feature values include one or more features of the evoked response data.

17. The method of claim 16, wherein the at least one stimulus comprises at least one auditory stimulus and/or at least one visual stimulus.

18. The method of claim 16, wherein processing the data comprises determining a percentage change in heart rate during and/or following delivery of the stimulus to the subject, based on the evoked cardiac activity data, and wherein the feature values include the percentage change in heart rate.

19. A non-transitory machine readable storage medium comprising instructions configured to cause a processor device to execute the method of claim 1.

20. A system for characterising tinnitus in a subject, the system comprising:
a processor device, configured to:
receive data, the received data comprising:
cortical activity data, indicative of cortical activity in one or more regions of the subject's brain, the cortical activity data derived from one or more functional near-infrared spectroscopy (fNIRS) signals; and
cardiac activity data, indicative of cardiac activity in the subject; and
process the received data, wherein the processing comprises:
inputting, into a model, feature values including one or more features of the cortical activity data and one or more features of the cardiac activity data,
wherein the model is configured to provide one or more classification results based on the feature values, the one or more classification results being indicative of at least one characteristic of tinnitus in the subject.

21. (canceled)

22. The system of claim 20, wherein the cardiac activity data is derived from at least one of:
the one or more fNIRS signals; or
one or more signals provided by one or more electrical sensors and/or one or more optical sensors.

23. (canceled)

24. The system of claim 22, wherein the one or more sensors comprises a photoplethysmography sensor.

25. The system of claim 20, wherein the fNIRS system comprises a multi-channel fNIRS system, wherein each channel is defined by a source-detector pair; or one or more channels configured to be positioned over each of the frontal, left and right temporal and occipital regions of the subject's brain.

26. (canceled)

27. The system of claim 20, comprising at least one of:
an auditory stimulator configured to deliver an auditory stimulus to the subject; or
a visual stimulator configured to deliver a visual stimulus to the subject.

28. (canceled)

29. The system of claim 20, wherein the model is a trained model, trained with an artificial intelligence (AI) algorithm.
